(19) United States
(12) Patent Application Publication
(10) Pub. No.: US 2002/0077322 A1
(43) Pub. Date: Jun. 20, 2002

(54) Protection of neurons against glutamate-induced damage in glaucoma and other conditions

(76) Inventor: George S. Ayoub, Santa Barbara, CA (US)

Correspondence Address: Michael B. Farber, Esq., OPPENHEIMER WOLFF & DONNELLY LLP, Suite 3800, 2029 Century Park East, Los Angeles, CA, 90067, US

(21) Appl. No.: 10/012,938

(22) Filed: Dec. 10, 2001

Related U.S. Application Data
(60) Provisional application No. 60/256,085, filed on Dec. 15, 2000.

(51) Int. Cl. A61K 31/5377; A61K 31/47; A61K 31/517; A61K 31/353; A61K 31/16

(52) U.S. Cl. 514/233.8; 514/266.3; 514/313; 514/416; 514/454; 514/627

(57) ABSTRACT

The present invention is directed to a method of protecting cells of the nervous system from glutamate-induced cytotoxicity , such as the type that is mimicked by administration of N-methyl-D-aspartate (NMDA), and which is associated with conditions such as ischemia or glaucoma. In general, the method comprises increasing the activity of a cannabinoid agonist that binds specifically to an endogenous cannabinoid receptor, such as the endogenous cannabinoid receptors CB₁ or CB₂, to protect the cells against glutamate-induced neurotoxicity. This can be done either by the administration of a cannabinoid agonist such as a physiologically acceptable salt of R(+)-[2,3-dihydro-5-methyl3-[(morpholinyl)methyl]pyrrolo [1,2,3-de]-1,4-benzoxazinyl]-(1-naphthalenyl) methanone, preferably the mesylate salt, or by blocking degradation of naturally-occurring endogenous cannabinoid agonists in the cells, such as by inhibition of anandamide amidohydrolase. Administration can be performed by one of several routes, such as enterally, transdermally, or transmucosally.

### BACKGROUND OF THE INVENTION

General Background and State of the Art: This invention is directed to methods of protecting neurons against glutamate-induced toxicity such as occurs in ischemic conditions, particularly as occurring in the eye disease glaucoma.

Although many advances have been made in the diagnosis and treatment of glaucoma, it remains a serious condition leading to a large number of cases of blindness. Glaucoma is characterized by an uncontrolled or uncontrollable increase in intraocular pressure. If not diagnosed and treated, as indicated, it can lead to blindness.

Glaucoma is a broad term encompassing a heterogeneous group of disorders affecting all age groups and linked by the common triad of increased intraocular pressure, cupping and atrophy of the optic nerve head, and visual field loss. Primary open angle glaucoma (POAG) is the most common form of glaucoma in the U.S., comprising about 60-70% of all adult cases. POAG is one of the leading causes of blindness in the U.S. Close relatives with patients with glaucoma have an increased risk. Other increased risk factors include diabetes, cardiovascular disease, elevated intraocular pressure, and myopia. POAG is insidious in onset and slowly progressive. Central vision is spared until late into the disease process, and therefore significant visual loss from glaucoma can occur without symptoms. Diagnosis consists of measuring the intraocular pressure, examining the optic disk, and testing the visual fields. POAG is typically caused by a relative obstruction to aqueous humor outflow from the trabecular meshwork. Medical treatment therefore includes agents that reduce the production of aqueous humor or facilitate non-trabecular aqueous outflow through uveoscleral pathway. A number of categories of drugs are available, including adrenergic agonists, β-adrenergic blocking agents, parasympathomimetic agents, carbonic anhydrase inhibitors, hyperosmotic agents, and prostaglandin analogues. Other forms of glaucoma exist, including secondary open angle glaucomas and primary and secondary angle closure glaucomas. Causes for these can include inflammation, tumors, pseudoexfoliation, and pigmentary glaucoma.

Glaucoma is one example of a condition in which damage to neurons can be caused by glutamate-induced neurotoxicity. Other examples are ischemic conditions and toxic conditions such as those mediated by N-methyl-D-aspartate (NMDA) type glutamate receptors, as well as other conditions that are characterized by endogenous glutamate released onto the cells.

Accordingly, there is therefore a need for improved methods of treating conditions that directly attack neurons and lead to cell death as a result of ischemic conditions and other conditions such as those associated with glutamate toxicity or with the administration of NMDA.

### INVENTION SUMMARY

The present invention provides methods for protecting the cells of the nervous system, such as ganglion cells or other cells of the central nervous system, from glutamate-induced neurotoxicity, such as occurs in glaucoma. In general, a method according to the present invention comprises increasing the activity of a cannabinoid agonist that binds specifically to an endogenous cannabinoid receptor to protect the cells of the nervous system, such as ganglion cells, against glutamate-induced neurotoxicity. More specifically, a method according to the present invention comprises increasing the activity of a cannabinoid agonist that binds specifically to either the CB₁ or CB₂ endogenous cannabinoid receptor.

In one alternative, the step of increasing the activity of the cannabinoid agonist comprises administering a cannabinoid agonist or analogue thereof to the cells. The cannabinoid agonist or analogue thereof is typically a cannabinoid analogue selected from the group consisting of anandamides, cannabinoids, pyrazole-4-carboxamides, benzamides, dihydroisoindolones, quinazolinediones, quinoline-carboxylic acid amides, dihydrooxazoles, and analogues and derivatives thereof. Preferably, the cannabinoid analogue is a physiologically acceptable salt of R(+)-[2,3-dihydro-5-methyl-3[(morpholinyl)methyl]pyrrolo [1,2,3-de]-1,4-benzoxazinyl]-(1-naphthalenyl) methanone. Most preferably, the physiologically acceptable salt is the mesylate (WIN 55212-2). The agonist can bind specifically to either the either the CB₁ or the CB₂ endogenous cannabinoid receptor.

In one preferred alternative, the cannabinoid agonist or analogue thereof is administered by a delivery mode such as transdermal delivery, transcleral delivery, intravitreal delivery, intravenous delivery, oral delivery, transmucosal delivery, or transepithelial delivery.

In another alternative, the step of increasing the activity of the cannabinoid agonist comprises blocking degradation of naturally-occurring endogenous cannabinoid agonists in the cells. This can occur by inhibition of anandamide amidohydrolase.

The protection of the cells against glutamate-induced cytotoxicity can protect the cells of the nervous system, including ganglion cells, against damage caused by glaucoma. Alternatively, the protection of the cells against glutamate-induced cytotoxicity can protect the cells of the nervous system, including ganglion cells, against damage caused by ischemic disease.

In another alternative, the protection of the cells against glutamate-induced cytotoxicity can protect the cells against damage caused by a disease or condition selected from the group consisting of epilepsy, grand mal seizures, global hypoxic ischemic insults, hypoxia, focal or global ischemia, Huntington's chorea, Parkinson's disease, Alzheimer's disease, hyperglycemia, traumatic injury, CNS trauma, stroke, cardiac arrest, diabetic retinopathy and other diabetic neuropathies, and macular degeneration.

In yet another alternative, the protection of the cells against glutamate-induced cytotoxicity can protect the cells against damage caused by a disease or condition selected from the group consisting of mental diseases, dementias, including AIDS dementia complex, inflammation, pain, schizophrenia, anorexia, multiple sclerosis, substance abuse, including but not limited to opioid, cocaine, and alcohol addiction, and spasticity.

In yet another alternative, a method for protecting against glutamate-induced neurotoxicity in cells of the central nervous system (CNS) comprising increasing the activity of a cannabinoid agonist that binds specifically to either the CB₁ or the CB₂ receptor to protect the cells of the central nervous system against glutamate-induced cytotoxicity.

In this alternative, the method can protect against damage caused by a disease or condition selected from the group consisting of stroke, hypoxia, focal or global ischemia, global hypoxic ischemic insults, Huntington's chorea, Parkinson's disease, Alzheimer's disease, hyperglycemia, diabetes, traumatic injury, CNS trauma, cardiac arrest, macular degeneration, mental diseases, schizophrenia, and anorexia.

### BRIEF DESCRIPTION OF THE DRAWINGS

The following invention will become better understood with reference to the specification, appended claims, and accompanying drawings where:

FIG. 1 is a graph showing results of counts of surviving ganglion cells in animals receiving saline injections in one eye, along with NMDA injections in the contralateral eye (Control =1; NMDA =2);

FIG. 2 is a graph showing results of counts of surviving ganglion cells in animals receiving NMDA injections in one eye, and NMDA plus WIN55212-2 injections in the contralateral eye (NMDA =1; NMDA + WIN =2);

FIG. 3 is a graph showing results of counts of surviving ganglion cells in animals receiving WIN55212-2 by topical application; the left pair of bars represents animals receiving saline injections in one eye and NMDA injections in the other eye; the right pair of bars represents animals receiving NMDA injections in each eye, and topical application of WIN55212-2 on one eye; numbers on the ordinate are cells/mm²; the data are means and standard errors from 9 mice (left pair of histograms) and 27 mice (right pair of histograms);

FIG. 4 is a four panel photomicrograph of FIG. 4 depicting examples from two pairs of mouse eyes; the top pair of photomicrographs are from one mouse that had received an injection of saline (A) and NMDA (B) into the right and left eyes, respectively, while the bottom pair are images from a second mouse that had received an injection of NMDA (C) and an injection of NMDA, followed by unilateral administration of topical WIN55212-2 (D); and

FIG. 5 is a graph showing counts of surviving retinal ganglion cells; the left pair of bars represents animals receiving NMDA injections in one eye and NMDA + WIN injections in the other eye (data taken from Example 1); the right pair of bars represents animals receiving NMDA + WIN injections in one eye, and NMDA, WIN and the CB₁ antagonist SR1 in the contralateral eye; numbers on the ordinate are cells/mm²; the data are means and standard errors from 9 mice (left pair of histograms) and 8 mice (right pair of histograms).

### DESCRIPTION

One of the consequences of exposure of neurons to ischemic conditions, such as occur in glaucoma and other conditions as discussed below, is the induction of glutamate-induced neurotoxicity. I have found a method for protecting directly against such neurotoxicity in ganglion cells, as well as in other cells of the nervous system, including other neurons. In general, a method according to the present invention comprises increasing the activity of a cannabinoid agonist that binds specifically to an endogenous cannabinoid receptor to protect the cells of the nervous system, such as ganglion cells, against glutamate-induced neurotoxicity. More specifically, a method according to the present invention comprises increasing the activity of a cannabinoid agonist that binds specifically to either the CB₁ or CB₂ endogenous cannabinoid receptor. In some applications, the increase of activity of a cannabinoid agonist that binds specifically to the CB₁ endogenous cannabinoid receptor is preferred.

In one embodiment of the method, the step of increasing the activity of the cannabinoid agonist comprises administering a cannabinoid agonist or analogue thereof to the cells. As indicated above, in some applications, the administration of a cannabinoid agonist that binds specifically to the CB₁ endogenous cannabinoid receptor is preferred.

Typically, the cannabinoid agonist is selected from the group consisting of anandamides, cannabinoids, pyrazole-4-carboxamides, benzamides, dihydroisoindolones, quinazolinediones, quinolone-carboxylic acid amides, dihydroxazoles, 3-alkyl-(5,5′-diphenyl) imidazolidinediones and analogues and derivatives thereof.

Among suitable analogues are the compound known as JWHO15, which is 2methyl-1-propyl-3-(1-naphthoyl)indole.

Suitable cannabinoid analogues are disclosed in U.S. Pat. No. 6,017,919 to Inaba et al., issued Jan. 25, 2000, incorporated herein by this reference. These compounds include the following acrylamide derivatives: (E)-N-[2-(4-hydroxyphenyl) ethyl]3-(4-methoxy-3-pentyloxyphenyl)-acrylamide; 3-(4-ethoxy-3-pentyloxyphenyl)-(E)-N-[2-(420736-11 hydroxyphenyl)ethyl]-acrylamide; 3-(3,4-dipentyloxyphenyl)-(E)-N-[2-(4hydroxyphenyl)ethyl]acrylamide; (E)-N-[2-(4-hydroxyphenyl) ethyl]-3-(4-methoxy-3butyloxyphenyl)-acrylamide; (E)-N-[2-(4-hydroxyphenyl) ethyl]-3-(4-methoxy-3hexyloxyphenyl)-acrylamide; (E)-N-[2-(4-hydroxyphenyl) ethyl]-3-(4-methoxy-3heptyloxyphenyl)-acrylamide; (E)-N-[2-(3-hydroxyphenyl) ethyl]-3-(4-methoxy-3pentyloxyphenyl )-acrylamide; (E)-N-[2-(2-hydroxyphenyl)ethyl]-3-(4-methoxy-3pentyloxyphenyl)-acrylamide; (E)-N-[2-(4-hydroxycyclohexyl)ethyl]-3-(4-methoxy-3pentyloxyphenyl)-acrylamide; (E)-N-[2-(4-hydroxyphenyl)ethyl]-N-methyl-3-( 4-methoxy-3pentyloxyphenyl)-acrylamide; (E)-N-[2-(4-hydroxyphenyl)ethyl]-3-(3-isopentyloxy-4methoxyphenyl)-acrylamide; 3-[3-(2-ethylbutyloxy)-4-methoxyphenyl]-(E)-N-[2-(4hydroxyphenyl)-ethyl]acrylamide; (E)-N-[2-(4-hydroxy-3-methoxyphenyl) ethyl ]-3-(4methoxy-3-pentyloxy-phenyl)acrylamide; 3-[3-(1,1-dimethylheptyl)-4-methoxyphenyl]-(E)-N[2-(4-hydroxyphenyl)-ethyl]acrylamide; (E)-N-[2-(3,4-dihydroxyphenyl) ethyl]-3-[3-(1,1dimethylheptyl)-4-methoxyphenyl]acrylamide; 3-(3-hexyl-4-methoxyphenyl)-(E)-N-[2(4hydroxyphenyl)ethyl]acrylamide; (E)-N-(4-amino-3-pentyloxyphenyl)-N-[2-(4hydroxyphenyl)ethyl]acrylamide; (E)-N-(4-amino-3-pentyloxyphenyl)-N-[2-(4nitrophenyl)ethyl]acrylamide; 3-(4-methoxy-3-pentyloxyphenyl)-(E)-N-[2-(4pentyloxyphenyl) ethyl]-acrylamide; (E)-N-[2-(4-methoxyphenyl) ethyl]-3-(4-methoxy-3-pentyloxyphenyl)-acrylamide; 3-(4-methoxy-3-pentyloxyphenyl)-(E)-N-(2morpholinoethyl)acrylamide; (E)-N-[2-(3,4-dihydroxyphenyl) ethyl]-3-(4-methoxy-3pentyloxyphenyl)-acrylamide; 2-[2-{3-(3-pentyloxy-4methoxyphenyl)acryloylamino}ethyl]pyridine-N-oxide; (E)-N-[2-(4-hydroxyphenyl) ethyl]-3-(4methoxy-3-pentylaminophenyl)-acrylamide; 3-[3-(N′, N′-dipentylamino)-4-methoxyphenyl](E)-N-[2-(4-hydroxyphenyl) ethyl]acrylamide; (E)-N-[2-(4-hydroxyphenyl) ethyl]-3-(3pentylamino-4-pentyloxyphenyl)-acrylamide; (E)-N-[2-(4-hydroxyphenyl) ethyl]-3-[3-(N′methyl-N′-pentylamino)-4-methoxyphenyl]acrylamide; (E)-N-[2-(4-hydroxyphenyl) ethyl]-3-(4methoxy-3-pentylthiophenyl)-acrylamide; (E)-N-[2-(4-hydroxyphenyl) ethyl]-3-(4-pentyloxy-3pentylthiophenyl)-acrylamide; (E)-N-[2-(4-aminophenyl) ethyl]-3-(4-methoxy-3pentyloxyphenyl)acrylamide; (E)-N-[2-(4-hydroxyphenyl) ethyl]-3-(3-pentyloxy-4pentylthiophenyl)-acrylamide; (E)-N-[2-(4-hydroxyphenyl) ethyl]-3-(3-pentyloxy-4methylthiophenyl)-acrylamide; (E)-N-[2-(4-aminophenyl) ethyl]-3-(4-methoxy-3-pentylthiophenyl)-acrylamide; (E)-N-[2-(4-nitrophenyl) ethyl]-3-(4-methoxy-3pentylthiophenyl)-acrylamide; (E)-N-[2-(imidazol-4-yl) ethyl]-3-(4-methoxy-3pentylthiophenyl)-acrylamide; (E)-N-[2-(4-nitrophenyl) ethyl]-3-(4-methoxy-3pentylaminophenyl)-acrylamide; (E)-N-[2-(imidazol-4-yl) ethyl]-3-(4-methoxy-3pentylaminophenyl)-acrylamide; (E)-N-[2-(4-hydroxyphenyl) ethyl]-3-(4-methylamino-3pentyloxy-phenyl)acrylamide; (E)-N-[2-(4-aminophenyl) ethyl]-3-(4-methoxy-3pentylaminophenyl)-acrylamide; (E)-N-[2-(4-nitrophenyl) ethyl]-3-(4-methylamino3pentyloxyphenyl)-acrylamide; 3-(4-methoxy-3-pentyloxyphenyl)-(E)-N-[2-(4-thiophen-2yl)ethyl]-acrylamide; (E)-N-[2-(4-hydroxyphenyl) ethyl]-3-[(N′-methyl-N′-pentylamino)-4pentyloxyphenyl]acrylamide; (E)-N-[2-(4-hydroxyphenyl) ethyl]-3-(4-pentylamino-3pentyloxyphenyl)-acrylamide; (E)-N-[2-(4-cyanophenyl) ethyl]-3-(4-methoxy-3pentyloxyphenyl)-acrylamide; and (E)-N-[2-(4-carbamoylphenyl) ethyl]-3-(4-methoxy-3pentyloxyphenyl)-acrylamide, and a pharmaceutically acceptable salt thereof.

Inaba et al. '919 also discloses benzamides, dihydroisoindolones, isoquinolinones, and quinazolinediones, as well as pentyloxyquinolines and dihydrooxazoles.

The benzamides include: N-[2-(4-hydroxyphenyl) ethyl]-4-methoxy-3pentyloxybenzamide; 4-ethoxy-N-[2-(4-hydroxyphenyl) ethyl]-3-pentyloxybenzamide; 3,4dipentyloxy-N-[2-(4-hydroxyphenyl)ethyl]benzamide; 4-dimethylamino-N-[2-(4hydroxyphenyl)ethyl]-3-pentyloxybenzamide; N-[2-(4-hyd roxyphenyl)ethyl]-3-pentylamino-4methoxybenzamide; 3-butyloxy-N-[2-(4-hydroxyphenyl) ethyl]-4-methoxybenzamide; 3hexyloxy-N-[2-(4-hydroxyphenyl) ethyl]-4-methoxybenzamide; 3-heptyloxy-N-[2-(4hydroxyphenyl) ethyl]-4-methoxybenzamide; N-[2-(3-hydroxyphenyl)ethyl]-methoxy-3pentyloxybenzamide; N-[2-(2-hydroxyphenyl) ethyl]-4-methoxy-3-pentyloxybenzamide; N-[2(4-hydroxycyclohexyl) ethyl]-4-methoxy-3-pentyloxybenzamide; N-[2-(4hydroxyphenyl) ethyl]-N-methyl-4-methoxy-3-pentyloxybenzamide; 3-isopentyloxy-N-[2-(4hydroxyphenyl)ethyl]-4-methoxybenzamide; 3-(2-ethylbutyloxy)-N-[2-(4hydroxyphenyl) ethyl]-4-methoxybenzamide; N-[2-(4-hydroxy-3-methoxyphenyl) ethyl]-4hydroxy-3-pentyloxybenzamide; N-[2-(4-hyd roxyphenyl) ethyl]-hydroxy-3pentyloxybenzamide; N-[2-(4-hydroxyphenyl) ethyl]-4-hydroxy-N-methyl-3pentyloxybenzamide; 3-(1,1-dimethylheptyl)-N-[2-(4-hydroxyphenyl)ethyl]-4methoxybenzamide; N-[2-(3,4-dihydroxyphenyl) ethyl]-3-(1,1-dimethylheptyl)-4methoxybenzamide; 3-(1 ,1-dimethylheptyl)-N-[2-(4-hydroxy-3-methoxyphenyl) ethyl]-4methoxybenzamide; 3-(1,-dimethylheptyl)-N-[2-(4-hydroxyphenyl) ethyl]-4hydroxybenzamide; N-[2-(3,4-dihydroxyphenyl) ethyl]-3-(1,1-dimethylheptyl)-4hydroxybenzamide; 3-hexyl-N-[2-(4-hydroxyphenyl) ethyl]-4-methoxybenzamide; N-[2-(4aminophenyl) ethyl]-3,4-dipentyloxybenzamide; 3 ,4-dihexyloxy-N-[2-(4hydroxyphenyl)ethyl]benzamide; 4-methoxy-N-[2-(4-pentyloxyphenyl) ethyl]-3pentyloxybenzamide; 4-methoxy-N-(2-morpholinoethyl)-3-pentyloxybenzamide; 4-methoxyN-[2-(4-propen-2-yloxyphenyl) ethyl]-3-pentyloxybenzamide; N-[2-(4-hydroxyphenyl) ethyl]methoxy-N-[2-(phenylsulfinyl) ethyl]-3-pentyloxybenzamide; N-[2-(3,4dihydroxyphenyl) ethyl]-4-methoxy-3-pentyloxybenzamide; N-[2-(4-acetoxyphenyl) ethyl]-4methoxy-3-pentyloxy-N-(E)-phenylthiovinylbenzamide; N-[2-(4-acetoxyphenyl) ethyl]-Nethyl-4-methoxy-3-pentyloxybenzamide; 4-[2-{N-(4-methoxy-3pentyloxybenzoyl)amino}ethyl]pyridine-N-oxide; 3-[2-{N-(4-methoxy-3pentyloxybenzoyl)amino}ethyl]pyridine-N-oxide; 3-d ipentylamino-N-[2-(4hydroxyphenyl)ethyl]methoxybenzamide; N-[2-(4-hydroxyphenyl)ethyl]-3-isohexyl-4methoxybenzamide; N-[2-(4-hydroxyphenyl) ethyl]-4-methoxy-3-(N′-methyl-N′-pentylamino)benzamide; N-[2-(4-hydroxyphenyl)ethyl]-3-pentylamino-4-pentyloxybenzamide; N-[2-(4-hydroxyphenyl) ethyl]-4-pentylamino-3-pentyloxybenzamide; 3 ,4-dipentyloxy-N-[2-(4sulfamoylphenyl)ethyl]benzamide; 3,4-dipentyloxy-N-[2-(imidazol-4-yl) ethyl]benzamide; 3,4pentyloxy-N-[2-(4-nitrophenyl)ethyl]benzamide; 3,4-dipentyloxy-N-[2-(4fluorophenyl)ethyl]benzamide; N-[2-(4-hydroxyphenyl) ethyl]-3-pentyloxy-4-propen-2ylbenzamide; N-[2-(4-hydroxyphenyl) ethyl]-4-propyloxy-3-pentyloxybenzamide; 3,4dibutyloxy-N-[2-(4-hydroxyphenyl)ethyl]-benzamide; 3 ,4-diheptyloxy-N-[2-(4hydroxyphenyl)ethyl]benzamide; N-[2-(4-hydroxyphenyl)ethyl]-4-methylamino-3pentyloxybenzamide; N-[2-(4-hydroxyphenyl)ethyl]-3,4-dipentylaminobenzamide; N-[2-(4hydroxyphenyl) ethyl]-3-(N′-methyl-N′-pentylamino)-4-pentyloxybenzamide; 4-amino-N-[2-(4hydroxyphenyl)ethyl]-3-pentyloxybenzamide; N-[2-(4-hydroxyphenyl)ethyl]-4-methoxy-3pentylthiobenzamide; N-[2-(4-hydroxyphenyl)ethyl]-4-pentyloxy-3-pentylthiobenzamide; 3,4dipentyloxy-N-[2-(2-thienyl)ethyl]benzamide; 3 ,4-dipentyloxy-N-[2-(5-hydroxyindol-3yl)ethyl]benzamide; 3,4-dipentyloxy-N-[2-(4-methylamiophenyl)ethyl]benzamide; N-[2-4-dimethylaminophenyl) ethyl]-3,4-dipentyloxybenzamide; 4-butyrylamino-N-[2-(4hydroxyphenyl) ethyl]-3-pentyloxybenzamide; N-[2-(4-hydroxyphenyl) ethyl]-4-formylamino-3pentylthiobenzamide; N-[2-(4-hydroxyphenyl)ethyl]-4-methylthio-3-pentyloxybenzamide; N[2-(4-hydroxyphenyl)ethyl]-3-pentyloxy-4-pentylthiobenzamide; N-[2-(4hydroxyphenyl) ethyl]-3-(4-hydroxybutyloxy)-4-methoxybenzamide, N-[2-4aminophenyl)ethyl]-4-methoxy-3-pentylthiobenzamide; 4-methoxy-N-[2-(4nitrophenyl)ethyl]-3-pentylthiobenzamide; N-[2-(imidazol-4-yl)ethyl]-4-methoxy-3pentylthiobenzamide; N-[2-(4-aminophenyl)ethyl]-4pentyloxy-3-pentylthiobenzamide; N-[2-(4-nitrophenyl)ethyl]-4-pentyloxy-3-pentylthiobenzamide; N-[2-(imidazol-4-yl)ethyl]-4pentyloxy-3-pentylthiobenzamide; and a pharmaceutically acceptable salt thereof.

The dihydroisoindolones include: 2-[2-(4-hydroxyphenyl) ethyl]-5-methoxy-4pentyloxy-2,3-dihydroisoindol-1-one; 2-[2-(4-benzyloxyphenyl) ethyl]-5-methoxy-4-pentyloxy2,3-dihydroisoindol-1-one; 5-methoxy-2-[2-(4-nitrophenyl) ethyl]-4-pentyloxy-2,3dihydroisoindol-1-one; 2-[2-(4-methyl phenyl) ethyl]-5-methoxy-4-pentyloxy-2 ,3dihydroisoindol-1-one; 4,5-dipentyloxy-2-[2-(imidazol-4-yl) ethyl]-2,3-dihydroisoindol-1-one; 2-[2-(4-benzyloxyphenyl) ethyl]-4,5-dipentyloxy-2 ,3-dihydroisoindol-1-one; 4,5-dipentyloxy-2[2-(4-nitrophenyl) ethyl]-2,3-dihydroisoindol-1-one; 2-[2-(4-aminophenyl) ethyl]-4,5dipentyloxy-2,3-dihydroisoindol- 1-one; 4,5-dipentyloxy-2-[2-(4-hydroxyphenyl) ethyl]-2,3dihydroisoindol-1-one; 4,5-dipentyloxy-2-[2-(4-methylaminophenyl ) ethyl]-2,3dihydroisoindol-1-one; 2-[2-(4-dimethylaminophenyl) ethyl]-4,5-dipentyloxy-2,3dihydroisoindol-1-one; 2-[2-(4-aminophenyl) ethyl]-5-methoxy-4-pentyloxy-2,3dihydroisoindol-1-one; 2-[2-(4-hydroxyphenyl) ethyl]-5-methoxy-4-pentylamino-2,3dihydroisoindol1-one; 5-methoxy-4-pentyloxy-2-[2-(4-pyridine) ethyl]-2,3-dihydroisoindol-1one; 2-[2-(4-dimethylaminophenyl) ethyl]-5-methoxy-4-pentyloxy-2,3-dihydroisoindol-1-one; 5-methoxy-2-[2-(4-methylaminophenyl) ethyl]-4-pentyloxy-2,3-dihydroisoindol-1-one, and a pharmaceutically acceptable salt thereof.

The isoquinolinones include 2-[2-(4-benzyloxyphenyl) ethyl]-6-methoxy-5-pentyloxy2H-isoquinolin-1-one; 2-[2-(4-hydroxyphenyl) ethyl]-6-methoxy-5-pentyloxy-2H-isoquinolin-1one; 2-[2-(4-pyridyl) ethyl]-6-methoxy-5-pentyloxy-2H-isoquinolin-1-one; 4-[2-(6-methoxy-1oxo-5-pentyloxy-1H-isoquinolin-2-yl) ethyl]phenyl acetate; 6-methoxy-2-[2-(4nitrophenyl) ethyl]-5-pentyloxy-2H-isoquinolin-1-one; 2-[2-(4-methylphenyl) ethyl]-6-methoxy5-pentyloxy-2H-isoquinolin-1-one; 6-methoxy-5-pentyloxy-2-(2-phenylethyl)-2H-isoquinolin1-one; 2-[2-(4-acetylaminophenyl) ethyl]-6-methoxy-5-pentyloxy-2H-isoquinolin-1-one; 5,6dipentyloxy-2-[2-(4-hydroxyphenyl) ethyl]-2H-isoquinolin-1-one; 2-[2-(4-aminophenyl) ethyl]6-methoxy-5-pentyloxy-2H-isoquinolin-1-one; 2-[2-(4-aminophenyl) ethyl]-6-methoxy-5pentyloxy-2H-isoquinolin-1-one hydrochloride; 2-[2-(4-dimethylaminophenyl) ethyl]-6methoxy-5-pentyloxy-2H-isoquinolin-1-one; 2-[2-(4-methylaminophenyl) ethyl]-6-methoxy-5pentyloxy-2H-isoquinolin-1-one; 6-methoxy-2-[2-(4-piperidinophenyl) ethyl]-5-pentyloxy-2H-isoquinolin-1-one; 6-methoxy-2-[2-(4-pyridyl) ethyl]-5-pentyloxy-2H-isoquinolin-1-one hydrochloride; 6-methoxy-2-[2-(4-oxocyclohexyl) ethyl]-5-pentyloxy-3,4-dihydro-2H-isoquinolin-1-one; 4-[2-(6-methoxy-1-oxo-5-pentyloxy-3,4-dihydro-1H-isoquinolin-2-yl)ethyl]phenyl acetate; 2-[2-(4-hydroxyphenyl)ethyl]-6-methoxy-5-pentyloxy-3 ,4-dihydro-2Hisoquinolin-1-one; 2-(2-phenylethyl)-6-methoxy-5-pentyloxy-3,4-dihydro-2H-isoquinolin-1one; 2-[2-(4-acetylaminophenyl)ethyl ]-6-methoxy-5-pentyloxy-3,4-dihydro-2H-isoquinolin-1one; 6-hydroxy-2-[2-(4-hydroxyphenyl)ethyl]-5-pentyloxy-3,4-dihydro-2H-isoquinolin-1-one; 2-[2-(4-methylphenyl) ethyl]-6-methoxy-5-pentyloxy-3,4-dihydro-2H-isoquinolin-1-one; 2-[2(4-aminophenyl)ethyl]-6-methoxy-5-pentyloxy-3,4-dihydro-2H-isoquinolin-1-one; 6-methoxy-5-pentyloxy-2-[2-(4-pyridyl)ethyl]-3,4-dihydro-2H-isoquinolin-1-one; 6-methoxy-1-oxo-5pentyloxy-3,4-dihydro-1H-isoquinolin-2-carboxylic acid N-(4-aminophenyl)amide; 6methoxy-1-oxo-5-pentyloxy-3,4-dihydro-1H-isoquinolin-2-carboxylic acid N-[(4aminophenyl)methyl]amide; 6-methoxy-1-oxo-5-pentyloxy-3,4-dihydro-1H-isoquinolin-2carboxylic acid N-(4-nitrophenyl)amide; and a pharmaceutically acceptable salt thereof.

The quinazolinediones include: 7-methoxy-3-[2-(4-nitrophenyl)ethyl]-8-pentyloxy(1H,3H)-quinazoline-2,4-dione; 7-methoxy-3-[2-(4-pyridyl )ethyl]-8-pentyloxy-(1H,3H)quinazoline-2,4-dione; 3-[2-(4-aminophenyl)ethyl]-7-methoxy-8-pentyloxy-(1H,3H)quinazoline-2 ,4-dione; 3-[2-(4-hydroxyphenyl) ethyl]-7-methoxy-8-pentyloxy-(1H,3H )quinazoline-2,4-dione; 3-[2-(4-methylaminophenyl)ethyl]-7-methoxy-8-pentyloxy-(1H,3H)quinazoline-2,4-dione; 3-[2-(4-dimethylaminophenyl)ethyl]-7-methoxy-8-pentyloxy-(1H,3H)quinazoline-2,4-dione; and a pharmaceutically acceptable salt thereof.

The pentyloxyquinolines include: 7-methoxy-8-pentyloxyquinoline-3-carboxylic acid N-[2-(4-pyridyl)-ethyl]amide; 7-methoxy-8-pentyloxyquinoline-3-carboxylic acid N-[2-(4hydroxy-phenyl)ethyl]amide; 7-methoxy-8-pentyloxyquinoline-3-carboxylic acid N-[2-(4aminophenyl)-ethyl]amide; 7-methoxy-8-pentyloxyquinoline-3-carboxylic acid N-[2-(4nitrophenyl)-ethyl]amide; 7-methoxy-8-pentyloxyquinoline-3-carboxylic acid N-[2-(imidazol 4-yl)ethyl]amide; and a pharmaceutically acceptable salt thereof.

The dihydrooxazoles include: 2-(4-methoxy-3-pentyloxyphenyl )-4,4-d imethyl-4,5dihydrooxazole; 2-(4-methoxy-3-pentylthiophenyl)-4,4-dimethyl-4,5-dihydrooxazole; 2-(3,4dipentyloxyphenyl)-4,4-dimethyl-4,5-dihydrooxazole; 2-(4-methylthio-3-pentyloxyphenyl)4,4-dimethyl4,5-dihydrooxazole; 2-(3-pentyloxy-4-pentylthiophenyl)-4,4-dimethyl-4,5dihydrooxazole; 2-(4-pentyloxy-3-pentylthiophenyl)-4,4-dimethyl-4,5-dihydrooxazole; 2-(4methoxy-3-pentyloxyphenyl)-5-(2-pyridyl)-4,5dihydrooxazole; and a pharmaceutically acceptable salt thereof.

U.S. Pat. No. 6,096,740 to Mechoulam et al., issued Aug. 1, 2000 (“Mechoulam et al. '740”), incorporated herein by this reference, discloses a number of cannabinoid analogues suitable for methods according to the present invention, including the (+)-(3S,4S)-1 ,1-dimethylheptyl homologue of 7-hydroxy-Δ⁶-tetrahydrocannabinol, also referred to as dexanabinol.

Also useful in methods according to the present invention are anandamide analogues as described in U.S. Pat. No. 5,977,180 to Pate et al. (“Pate et al. '180”), issued Nov. 2, 1999, and incorporated herein by this reference. These include arachidonyl propionitrileamide, arachidonyl ethanethiolamide, arachidonyl Δ-phenethylamide, arachidonyl N-acetylaminoethylamide, arachidonyl N,N,-dimethylaminoethylamide, arachidonyl aminoethylamide, arachidonyl pyridinoethylamide, arachidonyl morpholineamide, arachidonyl α-isopropylethanolamide, arachidonyl α-adimethylethanolamide, arachidonyl α-phenylethanolamide, arachidonyl α-isobutylethanolamide, and arachidonyl α- τ-butylethanolamide.

Other compounds suitable for use in methods according to the present invention as cannabinoid analogues are disclosed in U.S. Pat. No. 5,521,215 to Mechoulam et al. (“Mechoulam et al. '215”), issued May 28, 1996, and incorporated herein by this reference.

Additional compounds suitable for use in methods according to the present invention are the anandamides disclosed in U.S. Pat. No. 5,631,297 to Pate et al. (“Pate et al. '297”), issued May 20, 1997, incorporated herein by this reference. These compounds include arachidonyl ethanolamide, arachidonyl ethanethiolamide, arachidonylfluoroethylamide, 8,11,14-eicosatrienylethanolamide, arachidonyl propanolamide, 7,10,13,16-docosatetraenylethanolamide, palmitidyl ethanolamide, 4,7,10,13,16,1 9-docosahexaenylethanolamide, arachidyl fluoroethylamide, arachidonylamide, arachidonyl-1-methyl-ethanolamide, arachidonyl-2-methyl-ethanolamide, γ-linolenyl ethanolamide, and linoleyl ethanolamide.

Additional anandamides are N-2-hydroxyethyl)hexadecanamide, palmitoylethanolamide, which is an endogenous CB₂ agonist, as well as N-acylphosphatidylethanolamide.

Still other compounds suitable for use in methods according to the present invention are 3-alkyl-(5,5′-diphenyl)imidazolidinediones disclosed in M. Kanyonyo et al., “3Alkyl-(5,5′-diphenyl)imidazolidinediones as New Cannabinoid Receptor Ligands,” Bioorg. Med. Chem. Lett. 9: 2233-2236 (1999), incorporated herein by this reference. These include 3-ethylmorpholino-5,5′-di-ρ-bromophenylimidazolidinedione, 3-(1-hydroxypropyl)5,5′-di-ρ-bromophenylimidazolidinedione, and 3-heptyl-5,5′-di-ρ-bromophenylimidazolidinedione.

Other compounds include cannabinol analogues described in A. Mahadevan et al., “Novel Cannabinol Probes for CB1 and CB2 Cannabinoid Receptors,” J. Med. Chem. 43: 3778-3785 (2000), incorporated herein by this reference. These include 3-(1′,1′-dimethylheptyl) analogues, 9-substituted analogues, 11-hydroxy analogues, and deoxy analogues.

Still other compounds suitable for methods according to the present invention are cannabinoid analogues described in A. Buchwald et al., “Soft Cannabinoid Analogues as Potential Anti-Glaucoma Agents,” Pharmazie 55: 196-201 (2000). These compounds include compounds with esters or reverse esters incorporated into the side chain of the compound 5,5-dimethyl-8-(1,2-dimethylheptyl)-10-hydroxy-2-2(2-propynyl)-1,2,3,4tetrahydro-5H-[1]benzopyrano[3,4-d]pyridine.

Still other compounds suitable for methods according to the present invention are analogues of tetrahydrocannabinol disclosed in M. Szirmai & M. M. Haldin, “A Urinary Metabolite of Δ¹-Tetrahydrocannabinol. The First Synthesis of 4″,5″-Bisnor-Δ¹Tetrahydrocannabinol-7,3″-Dioic Acid,” Bioorg Med. Chem. 3: 899-906 (1995), incorporated herein by this reference. These compounds include the tetrahydrocannabinol analogue 4″,5″-bisnor-Δ¹-tetrahydrocannabinol-7,3″-dioic acid.

Still other cannabinoid analogues are those disclosed in Keimowitz, jm9902281, J. Med. Chem. 1999, which include cannabinoid analogues with aliphatic side chains, such as heptynyl, heptenyl, octynyl, octenyl, bromohexynyl, bromohexenyl, nonynyl, and other side chains with double or triple bonds.

Still other cannabinoid analogues include those disclosed in A. J. Hampson et al., “Cannabidiol and (−)Δ⁹-Tetrahydrocannabinol are Neuroprotective Antioxidants,” Proc. Natl. Acad. Sci. U.S.A., 95: 8268-8273 (1998), and L. L. Iversen, “The Science of Marijuana” (Oxford University Press, 2000), pp.40, 42-43, 60, both of which are hereby incorporated by this reference.

Other suitable compounds are known in the art. A particularly suitable cannabinoid agonist is a physiologically acceptable salt of R(+)-[2,3-dihydro-5-methyl-3[(morpholinyl)methyl]pyrrolo[1,2,3-de]-1,4-benzoxazinyl]-1-naphthalenyl)methanone. Preferably, the salt is the mesylate. A suitable mesylate salt (WIN 55212-2) is available from Sigma-RBI (St. Louis, Mo.).

Other compounds include (+) 3S,4S-5′-(-Δ-butyl)-7-hydroxy-Δ⁶-tetrahydrocannabinol, which is believed to inhibit NMDA receptors, 5′-(1′,1′-dimethylbutyl)7-hydroxy-Δ⁶-tetrahydrocannabinol, known as CP59940, which acts on CB₁ and CB₂, 3-[2hydroxy-4-(1,1-dimethylheptyl)phenyl]-4-(3-hydroxypropyl)cyclohexan-1-ol, arachidonylcyclopropylamide (“ACPA”), which acts on CB₁, arachidonyl-2-chloroethylamide (“ACEA”), which acts on CB₁, (6aR, 10aR)-3-(1,1-dimethylheptyl)-1-methoxy)-6,6-dimethyl9-methylene-6a ,7,8,9,10,10 a-hexahydro-6H-benzo[c]chromene, known as L759656, which acts on CB₂, (6aR, 10aR)-3-(1, 1-dimethylheptyl)-1-methoxy)-6,6,9-trimethyl-6a ,7,1 0,10 atetrahydro-6H-benzo[c]chromene, known as L759633, which also acts on CB₂, and bicyclo [3.1.1]hept-2-ene-2-methanol-4-[4(1,1-dimethylheptyl)-2,6-dimethyloxyphenyl-6,6-dimethyl (1R,4R,5R).

Still other compounds that are useful in methods according to the present invention are known in the art.

The cannabinoid agonist can be administered by a number of routes. Depending upon the particular needs of the individual subject involved, the compositions used in the present invention can be administered in varying doses to provide effective treatment concentrations based upon the teachings of the present invention. What constitutes an effective amount of the selected composition will vary based upon such factors as the activity of the selected cannabinoid agonist or enzyme inhibitor, the physiological characteristics of the subject, the extent and nature of the subject's disease or condition and the method of administration. Generally, initial doses will be modified to determine the optimum dosage for treatment of the particular mammalian subject. The compositions can be administered using a number of different routes including orally, topically, transdermally, transclerally, transepithelially, intraocularly, intravitreally, enteral administration, administration by intraperitoneal injection or administration by intravenous injection directly into the bloodstream. The compositions to be used can also be administered via transmucosal application, such as by a nasal spray, inhaler, or by sublingual application. Of course, effective amounts of the cannabinoid agonists or enzyme inhibitors can also be administered through injection into the cerebrospinal fluid or infusion directly into the brain, if desired. A particularly preferred administration route is transdermally.

The methods of the present invention can be effected using cannabinoid agonists or enzyme inhibitors administered to a mammalian subject either alone or in combination as a pharmaceutical formulation. Further, the cannabinoid agonists or enzyme inhibitors can be combined with pharmaceutically acceptable excipients and carrier materials such as inert solid diluents, aqueous solutions, or non-toxic organic solvents. If desired, these pharmaceutical formulations can also contain preservatives and stabilizing agents and the like, as well as minor amounts of auxiliary substances such as wetting or emulsifying agents, as well as pH buffering agents and the like which enhance the effectiveness of the active ingredient. The pharmaceutically acceptable carrier can be chosen from those generally known in the art including, but not limited to, human serum albumin, ion exchangers, dextrose, alumina, lecithin, buffer substances such as phosphate, glycine, sorbic acid, potassium sorbate, propylene glycol, polyethylene glycol, and salts or electrolytes such as protamine sulfate, sodium chloride, or potassium chloride. Other carriers can be used.

Liquid compositions can also contain liquid phases either in addition to or to the exclusion of water. Examples of such additional liquid phases are glycerin, vegetable oils such as cottonseed oil, organic esters such as ethyl oleate, and water-oil emulsions.

The compositions can be made into aerosol formulations (i.e., they can be “nebulized”) to be administered via inhalation. Aerosol formulation can be placed into pressurized acceptable propellants, such as dichloromethane, propane, or nitrogen. Other suitable propellants are known in the art.

Formulations suitable for enteral administration include aqueous and non-aqueous, isotonic sterile solutions. These can contain antioxidants, buffers, preservatives, bacteriostatic agents, and solutes that render the formulation isotonic with the blood or fluid of the particular recipient as required. Alternatively, these formulations can be aqueous or non-aqueous sterile suspensions that can include suspending agents, thickening agents, solubilizers, stabilizers, and preservatives. Preparation of solutions for enteral administration is well known in the art and need not be described further here.

Formulations suitable for parenteral administration, such as, for example, by intravenous, intraocular, intravitreal, intramuscular, intradermal, and subcutaneous routes, include aqueous and non-aqueous, isotonic sterile injection solutions. These can contain antioxidants, buffers, preservatives, bacteriostatic agents, and solutes that render the formulation isotonic with the blood or fluid of the particular recipient as required. Alternatively, these formulations can be aqueous or non-aqueous sterile suspensions that can include suspending agents, thickening agents, solubilizers, stabilizers, and preservatives. Compositions suitable for use in methods according to the present invention could be administered, for example, by intravenous infusion, orally, topically, transdermally, intraocularly, intravitreally, transepithelially, transsclerally, intraperitoneally, intravesically, or intrathecally. Formulations of compounds suitable for use in methods according to the present invention can be presented in unit-dose or multi-dose sealed containers, in physical form such as ampoules or vials.

Another embodiment of the present invention is a method for protecting against glutamate-induced neurotoxicity in ganglion cells where the step of increasing the activity of the cannabinoid agonist comprises blocking of degradation of naturally-occurring endogenous cannabinoid agonists in the cells. Typically, these naturally-occurring endogenous cannabinoid agonists in the cells are anandamides, and the blockage of the naturally-occurring endogenous cannabinoid agonists occurs by inhibition of anandamide amidohydrolase. Methods and compositions for blocking anandamide amidohydrolase, also known as anandamide amidase, are disclosed in U.S. Pat. No. 5,874,459 to Makriyannis et al. (“Makriyannis et al. '459”), issued Feb. 23, 1999, and incorporated herein by this reference. The inhibitors disclosed in Makriyannis et al. '459 include arachidonylethanolamide, palmitylsulfonyl fluoride, other sulfonyl fluorides, N[(alkylsulfonyl)oxy]succinimides and N-O-diacylhydroxylamines.

Additional anandamide amidase inhibitors are disclosed in U.S. Pat. No. 5,688,825 to Makriyannis et al. (“Makriyannis et al. '825”), issued Nov. 18, 1997, and incorporated herein by this reference. Still other anandamide aminohydrolase inhibitors are disclosed in U.S. Pat. No. 5,925,672 to Piomelli et al. (“Piomelli et al. '672”) issued Jul. 20, 1999, and incorporated herein by this reference. Piomelli et al. '672 discloses hallenol lactones such as E-6-(bromomethylene)tetrahydro-3-(1-naphthalenyl)-2H-pyrane-2-one.

Other inhibitors of anandamide hydrolysis can also be used.

Methods of the present invention can be used to treat or prevent neural damage due to ischemia, glaucoma, and a number of nervous system diseases such as epilepsy, grand mal seizures, global hypoxic ischemic insults, hypoxia, focal or global ischemia, Huntington's chorea, Parkinson's disease, Alzheimer's disease, dementias, including AIDS dementia complex, hyperglycemia, traumatic injury, CNS trauma, stroke, cardiac arrest, diabetic retinopathy, macular degeneration, as well as mental diseases, inflammation, pain, schizophrenia, anorexia, multiple sclerosis, substance abuse, including but not limited to opioid, cocaine, and alcohol addiction, and spasticity.

In a particular application, methods according to the present invention can be used to protect cells of the central nervous system against glutamate-induced neurotoxicity, particularly against a disease or condition selected from the group consisting of stroke, hypoxia, focal or global ischemia, global hypoxic ischemic insults, Huntington's chorea, Parkinson's disease, Alzheimer's disease, hyperglycemia, diabetes, traumatic injury, CNS trauma, cardiac arrest, macular degeneration, mental diseases, schizophrenia, and anorexia.

The invention is illustrated by the following Examples. These Examples are presented for illustrative purposes only and is not intended to limit the invention.

### Example 1

### Effect of WIN Mesylate on Protecting NMDA-Treated Ganglions from Cell Loss

### Procedure

Experiments were performed on adult C57/BL6 mice of an average of 4 months, weighing 15-25 g. Animals were given food and water ad libitum. All studies were conducted in accordance with the principles and procedures outlined in the National Institute of Health (NIH) Guide for the Care and Use of Laboratory Animals.

Three sterile solutions were administered by intravitreal injection: balanced saline solution, NMDA (Sigma) and NMDA plus WIN 55,212-2 (Sigma). The NMDA solution was comprised of 320 mM NMDA in balanced saline solution (net of 160 nmol of NMDA injected per eye). The WIN solution contained 0.5 mM WIN 55,212-2 in the NMDA solution using a DMSO vehicle (less than 0.1% of solution) to carry WIN (net of 0.25 nmol of WIN injected per eye).

Intravitreal Injections Mice were anaesthetized by intraperitoneal injection of 0.017 ml/g body weight of a solution containing 1.75% tribromoethanol and 1.75% tertiary amyl alcohol. A topical application of 0.5% proparacaine hydrochloride was administered prior to intravitreal injections. A small incision was made with a 22-gauge needle in the dorsal limbus, through the conjunctiva and sclera. A Hamilton syringe was passed through this incision at a 40 to 50 degree angle to the equator to administer the solution. All eyes were injected with 0.5 μl of solution. All procedures were performed under microscopy. Occasionally this procedure produced cataracts in mice (less than 5%) due to damage to the lens with the syringe. These mice were not used in the analysis.

Tissue Preparation Flat mount preparations of the retinae and counts of cells in the retinal ganglion cell layers were performed to evaluate cell loss. Ten days after intravitreal injections, animals were euthanized by an intraperitoneal overdose injection of pentobarbital. Animals were immediately perfused with 4% paraformaldehyde, and the eyes enucleated. The retinae were dissected and mounted onto gelatinized slides, ganglion cell layer up. Several radial cuts were made at the periphery, and the surface was cleared and flattened with a fine brush (camel hair, size 0). The flat mounted retinae were immersed overnight in a solution containing 10% formaldehyde and 90% alcohol. The sections were subsequently dehydrated and stained with 0.5-1% cresyl violet and enclosed with coverslips.

Morphometric Analysis (Quantification of Cell Loss in the Retinal Ganglion Cell Layer) The ganglion cell layer was imaged at 400×and cells were counted within 70×70 um grids. Samples taken were located midway between the optic nerve head and periphery in four quadrants of the flat mounted retinae (nasal, temporal, superior, inferior). Quandrant counts from each retina were averaged to give the value for each eye. Morphologically distinguishable glial cells and vascular endothelial cells were excluded from the cell count, but no attempt was made to distinguish between ganglion and amacrine cells.

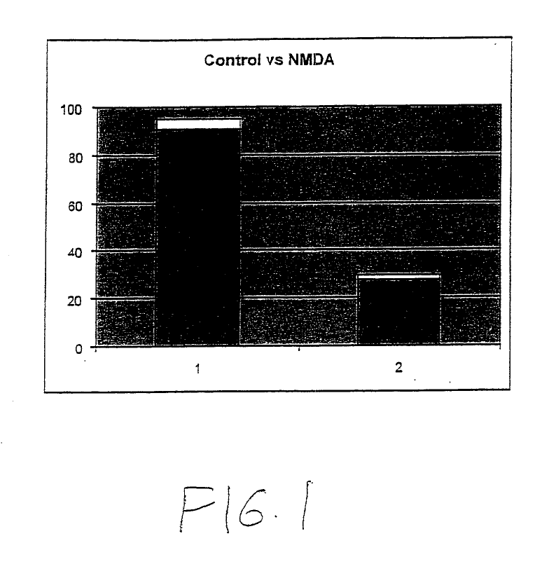

The results of counts of surviving ganglion cells in animals receiving saline injections in one eye, along with NMDA injections in the contralateral eye are shown in FIG. 1. Values represent the means and standard deviations of data from 9 mice. (Control=1; NMDA=2).

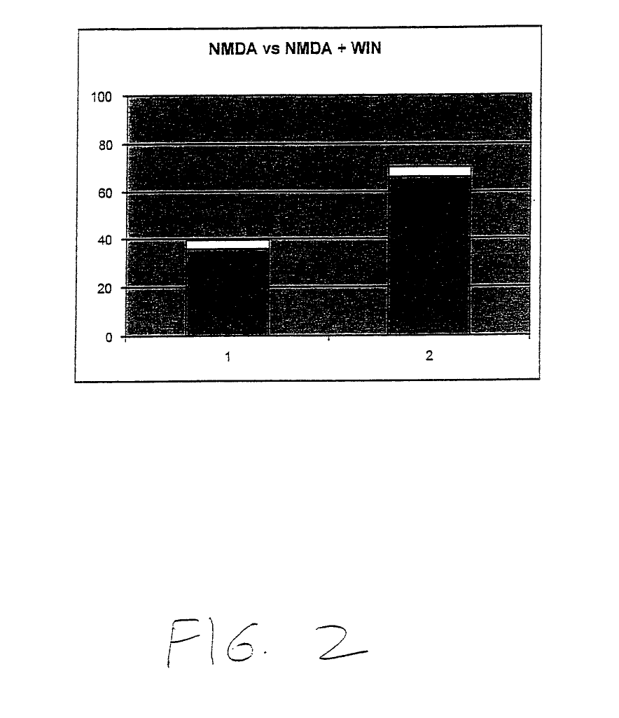

The results of counts of surviving ganglion cells in animals receiving NMDA injections in one eye, and NMDA plus WIN injections in the contralateral eye are shown in FIG. 2. Values represent the means and standard deviations of data from 15 mice (NMDA=1; NMDA+WIN=2)

### Analysis

NMDA caused a 70% loss of retinal ganglion cells in the 10 day period. A single application of WIN diminished this loss by 80%, resulting in only a 30% loss of ganglion cells in the same period.

Activation of cannabinoid receptors via WIN thus provides protection from NMDA induced damage to neurons.

### Example 2

### Effect of Topical Application of WIN Mesylate on Protecting NMDA-Treated Retinal Ganglion Cells from Cell Death

### Procedure

Experiments were performed on adult C57/BL6 mice of an average of 4 months, weighing 15-25 g. Animals were given food and water ad libitum. All studies were conducted in accordance with the principles and procedures outlined in the National Institute of Health (NIH) Guide for the Care and Use of Laboratory Animals.

Two sterile solutions were administered by intravitreal injection: balanced saline solution, and NMDA (Sigma). The NMDA solution was comprised of 320 mM NMDA in balanced saline solution (net of 160 nmol of NMDA injected per eye).

Intravitreal Injections: Mice were anaesthetized by intraperitoneal injection of 0.017 ml/g body weight of a solution containing 1.75% tribromoethanol and 1.75% tertiary amyl alcohol. A topical application of 0.5% proparacaine hydrochloride was administered prior to intravitreal injections. A small incision was made with a 22-gauge needle in the dorsal limbus, through the conjunctiva and sclera. A Hamilton syringe was passed through this incision at a 40 to 50 degree angle to the equator to administer the solution. All eyes were injected with 0.5μl of solution. All procedures were performed under microscopy. Occasionally this procedure produced cataracts in mice (less than 5%) due to damage to the lens with the syringe. These mice were not used in the analysis.

Topical Application of cannabinoid: A topical application solution of WIN55212-2 (Sigma) was prepared in 2-hydroxypropyl-β-cyclodextrin (Sigma). This solution contained 2.4 mg of WIN55212-2 in 1 ml 2-hydroxypropyl-β-cyclodextrin.

10 μl of this WIN55212-2 solution was applied to the anterior surface of the eye. In the experiments described, it was applied three times daily on one eye only. The applications of the WIN solution began one day after the NMDA injections were made, to allow time for the surface of the eye to heal, and the topical applications were made for 9 days. Thus, while each eye received the NMDA injections, only one eye of each animal had the WIN solution applied onto it.

Tissue Preparation: Flat mount preparations of the retinae and counts of cells in the retinal ganglion cell layers were performed to evaluate cell loss. Ten days after intravitreal injections, animals were euthanized by an intraperitoneal overdose injection of pentobarbital. Animals were immediately perfused with 4% paraformaldehyde, and the eyes enucleated. The retinae were dissected and mounted onto gelatinized slides, ganglion cell layer up. Several radial cuts were made at the periphery, and the surface was cleared and flattened with a fine brush (camel hair, size 0). The flat mounted retinae were immersed overnight in a solution containing 10% formaldehyde and 90% alcohol. The sections were subsequently dehydrated and stained with 0.5-1% cresyl violet and enclosed with coverslips.

Morphometric Analysis: The ganglion cell layer was imaged at 400X and 630X and cells were counted within 120×120 μm square grids. Four samples were taken at retinal loci midway between the optic nerve head and the retinal periphery in each quadrant of the flat mounted retinae (nasal, temporal, superior, inferior). Quadrant counts from each retina were averaged to give the value for each eye. Morphologically distinguishable glial cells and vascular endothelial cells were excluded from the cell count, but no attempt was made to distinguish between ganglion and amacrine cells.

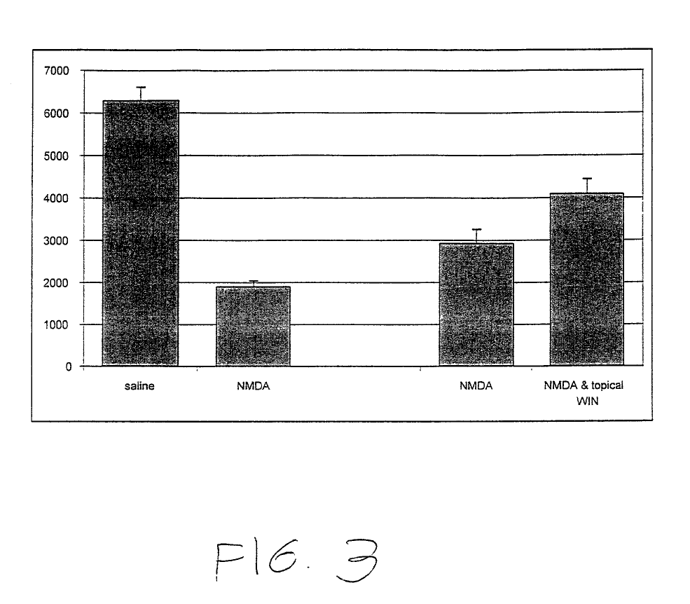

FIG. 3: Counts of surviving retinal ganglion cells. The left pair of bars represents animals receiving saline injections in one eye and NMDA injections in the other eye. The NMDA decreased the number of surviving ganglion cells by 70%. The right pair of bars represents animals receiving NMDA injections in each eye, and topical application of WIN55212-2 on one eye. Topical application of WIN protected about 50% of the retinal ganglion cells in the treated eye. In the contralateral eye (to which no WIN was directly applied), there was a smaller amount of protection provided (protecting about 25% of the cells), presumably from systemic delivery of WIN that had been applied to the other eye. Numbers on the ordinate are cells/mm². The data are means and standard errors from 9 mice (left pair of histograms) and 27 mice (right pair of histograms).

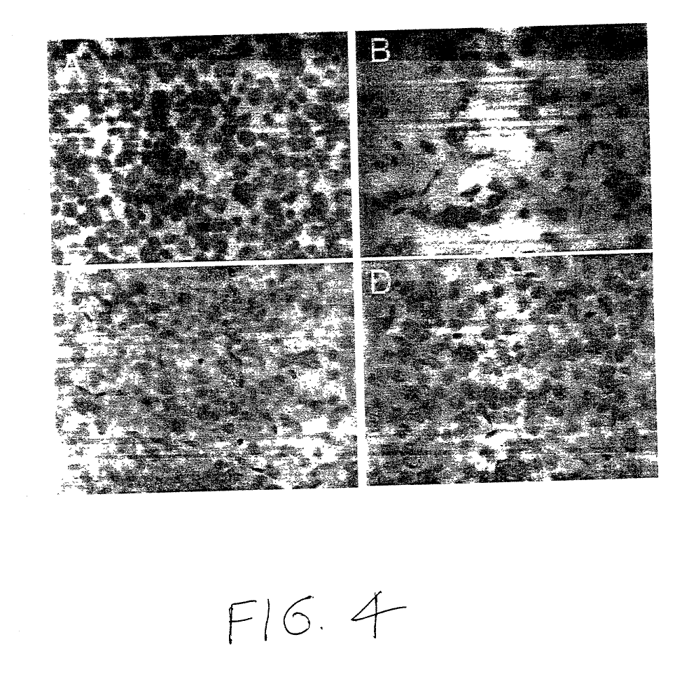

The four panel photomicrograph of FIG. 4 depicts examples from two pairs of mouse eyes. The top pair of photomicrographs are from one mouse that had received an injection of saline (A) and NMDA (B) into the right and left eyes, respectively, while the bottom pair are images from a second mouse that had received an injection of NMDA (C) and an injection of NMDA, followed by unilateral administration of topical WIN55212-2 (D). A comparison of (A) and (B) reveals a substantial reduction in the density of neurons within the ganglion cell layer following NMDA-treatment. Neurons of all sizes were affected. A comparison of (C) and (D) shows that the coincident exposure of the NMDA-treated retina to WIN55212-2 ameliorated the excitotoxic damage produced by this glutamate analogue.

### Analysis

NMDA caused a 70% loss of retinal ganglion cells in the 10 day period. Topical application of WIN on days 2-9 diminished this loss by approximately 50%, resulting in only a 30% loss of ganglion cells in the same period. Additionally, the contralateral eye (the eye to which WIN was not applied) received some protection from the applied WIN, presumably from systemic delivery of the drug. In this contralateral eye, about 25% of affected ganglion cells were protected.

Thus, topical delivery of cannabinoid agonists is able to activate retinal cannabinoid receptors, and thus provide protection from NMDA induced damage to neurons.

Additionally, the data indicate that indirect application of cannabinoid agonists (as in the contralateral eye) is able to provide protection from NMDA induced damage to neurons. Thus, application of WIN topically to the surface of the eye, or systemically provides a level of protection for retinal ganglion cells from NMDA damage.

This has implications for neural damage due to ischemia, anoxia, glaucoma, diabetic retinopathy, and a number of nervous system diseases mentioned previously.

### Example 3

### Effect of Cannbinoid Antagonists on WIN Protection of NMDA-Treated Retinal Ganglion Cells

### Procedure

Experiments were performed on adult C57/BL6 mice of an average of 4 months, weighing 15-25 g. Animals were given food and water ad libitum. All studies were conducted in accordance with the principles and procedures outlined in the National Institute of Health (NIH) Guide for the Care and Use of Laboratory Animals.

Sterile solutions were administered by intravitreal injection: NMDA (Sigma) and WIN 55212-2 (Sigma), along with the selective CB₁ cannabinoid antagonist, AM 251 (Tocris Cookson). This antagonist is structurally similar to SR 141716A. The NMDA solution was comprised of 320 mM NMDA in balanced saline solution (net of 160 nmol of NMDA injected per eye), WIN was used at a concentration of 0.5 mM, injecting 0.25 nmol per eye, and AM 251 was used at one-half the dosage of WIN.

Intravitreal Injections: Mice were anaesthetized by intraperitoneal injection of 0.017 ml/g body weight of a solution containing 1.75% tribromoethanol and 1.75% tertiary amyl alcohol. A topical application of 0.5% proparacaine hydrochloride was administered prior to intravitreal injections. A small incision was made with a 22-gauge needle in the dorsal limbus, through the conjunctiva and sclera. A Hamilton syringe was passed through this incision at a 40 to 50 degree angle to the equator to administer the solution. All eyes were injected with 0.5 μl of solution. All procedures were performed under microscopy. Occasionally this procedure produced cataracts in mice (less than 5%) due to damage to the lens with the syringe. These mice were not used in the analysis.

Each mouse received an intraocular injection of NMDA plus WIN in one eye, and received an injection of NMDA, WIN, and the CB₁ cannabinoid antagonist (AM 251) in the contralateral eye.

Tissue Preparation: Flat mount preparations of the retinae and counts of cells in the retinal ganglion cell layers were performed to evaluate cell loss. Ten days after intravitreal injections, animals were euthanized by an intraperitoneal overdose injection of pentobarbital. Animals were immediately perfused with 4% paraformaldehyde, and the eyes enucleated. The retinae were dissected and mounted onto gelatinized slides, ganglion cell layer up. Several radial cuts were made at the periphery, and the surface was cleared and flattened with a fine brush (camel hair, size 0). The flat mounted retinae were immersed overnight in a solution containing 10% formaldehyde and 90% alcohol. The sections were subsequently dehydrated and stained with 0.5-1% cresyl violet and enclosed with coverslips.

Morphometric Analysis: The ganglion cell layer was imaged at 400X and 630X and cells were counted within 120×120 μm square grids. Four samples were taken at retinal loci midway between the optic nerve head and the retinal periphery in each quadrant of the flat mounted retinae (nasal, temporal, superior, inferior). Quadrant counts from each retina were averaged to give the value for each eye. Morphologically distinguishable glial cells and vascular endothelial cells were excluded from the cell count, but no attempt was made to distinguish between ganglion and amacrine cells.

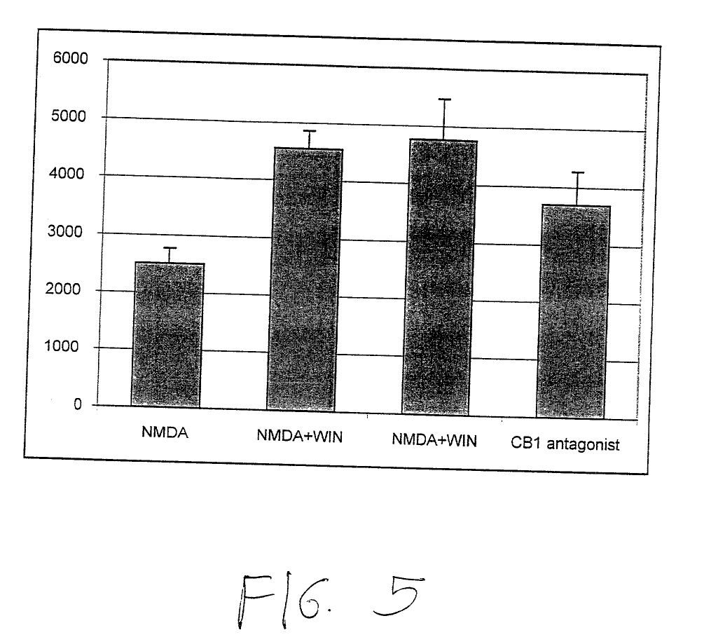

FIG. 5 shows counts of surviving retinal ganglion cells. The left pair of bars represents animals receiving NMDA injections in one eye and NMDA+WIN injections in the other eye (data are taken from Example 1). The WIN increased the number of surviving ganglion cells by a factor of two. The right pair of bars represents animals receiving NMDA +WIN injections in one eye, and NMDA, WIN and SR1 in the contralateral eye. The CB₁ antagonist partially blocked the effect of WIN in protecting the retinal ganglion cells from NMDA. Numbers on the ordinate are cells mm². The data are means and standard errors from 9 mice (left pair of histograms) and 8 mice (right pair of histograms).

### Analysis

WIN protects a number of retinal ganglion cells from NMDA in the 10 day period. Co-application of the CB₁ antagonist (at dose equivalent to its Kᵢ) along with WIN blocked about half the protective effect of WIN, consistent with the effect of WIN being mediated via a CB₁ cannabinoid receptor.

Preliminary tests of the CB₂ antagonist, AM 630 (Tocris Cookson) in combination with WIN and NMDA revealed the same cell counts as the WIN+NMDA eye, indicating that the WIN effect is likely not via the CB₂ cannabinoid receptor.

This has implications for neural damage due to ischemia, anoxia, glaucoma, diabetic retinopathy, and a number of nervous system diseases mentioned previously.

### DETAILED DESCRIPTION OF THE PREFERRED EMBODIMENTS

### ADVANTAGES OF THE PRESENT INVENTION

Methods according to the present invention provide protection of ganglion cells in the retina and other tissues that would otherwise be damaged by glutamate-induced neurotoxicity caused by glutamate, NMDA, or other toxic agents, or by ischemia, hypoxia, or other environmental insults, as well as protecting other cells of the nervous system, particularly the central nervous system. Methods according to the present invention provide direct protection to ganglion cells and thus open a new route for the treatment of glaucoma in addition to presently-available methods for reducing intraocular pressure. Methods according to the present invention have the advantage that they protect ganglion cells from cell death and thus avert consequences of glaucoma stemming from such cell death.

Although the present invention has been described in considerable detail, with reference to certain preferred versions thereof, other versions and embodiments are possible. Therefore, the scope of the invention is determined by the following claims.

While the specification describes particular embodiments of the present invention, those of ordinary skill can devise variations of the present invention without departing from the inventive concept.

We claim:

1. A method for protecting against glutamate-induced neurotoxicity in cells of the nervous system comprising increasing the activity of a cannabinoid agonist that binds specifically to an endogenous cannabinoid receptor to protect the cells against glutamate-induced neurotoxicity.

2. The method of claim 1 wherein the endogenous cannabinoid receptor is either the CB₁ or CB₂ endogenous cannabinoid receptor.

3. The method of claim 2 wherein the endogenous cannabinoid receptor is the CB₁ endogenous cannabinoid receptor.

4. The method of claim 2 wherein the endogenous cannabinoid receptor is the CB₂ endogenous cannabinoid receptor.

5. The method of claim 2 wherein the step of increasing the activity of the cannabinoid agonist comprises administering a cannabinoid agonist or analogue thereof to the cells.

6. The method of claim 5 wherein the cannabinoid agonist or analogue thereof is a cannabinoid analogue selected from the group consisting of anandamides, cannabinoids, pyrazole-4-carboxamides, benzamides, dihydroisoindolones, quinazolinediones, quinolinecarboxylic acid amides, dihydrooxazoles, and analogues and derivatives thereof.

7. The method of claim 6 wherein the cannabinoid agonist is a physiologically acceptable salt of R(+)-[2 ,3-dihyd ro-5-methyl-3-[(morpholinyl)methyl]pyrrolo [1 ,2,3-de]-1 ,4benzoxazinyl]-(1-naphthalenyl) methanone.

8. The method of claim 7 wherein the salt is the mesylate.

9. The method of claim 5 wherein the cannabinoid agonist or analogue thereof is administered by transdermal delivery.

10. The method of claim 5 wherein the cannabinoid agonist or analogue thereof is administered by transscleral delivery.

11. The method of claim 5 wherein the cannabinoid agonist or analogue thereof is administered by transmucosal delivery.

12. The method of claim 11 wherein the transmucosal delivery is performed by the use of a nasal spray, by the use of an inhaler, or by sublingual application of the agonist.

13. The method of claim 1 wherein the cells are ganglion cells.

14. The method of claim 1 wherein the protection of the cells against glutamate-induced cytotoxicity protects the cells against damage caused by glaucoma.

15. The method of claim 1 wherein the protection of the cells against glutamate-induced cytotoxicity protects the cells against damage caused by ischemic disease.

16. The method of claim 1 wherein the protection of the cells against glutamate-induced cytotoxicity protects the cells against damage caused by a disease or condition selected from the group consisting of epilepsy, grand mal seizures, global hypoxic ischemic insults, hypoxia, focal or global ischemia, Huntington's chorea, Parkinson's disease, Alzheimer's disease, hyperglycemia, traumatic injury, CNS trauma, stroke, cardiac arrest, diabetic retinopathy, and macular degeneration.

17. The method of claim I wherein the protection of the cells against glutamate-induced cytotoxicity protects the cells against damage caused by a disease or condition selected from the group consisting of mental diseases, AIDS dementia complex, other dementias, inflammation, pain, schizophrenia, anorexia, multiple sclerosis, opioid addiction, cocaine addiction, alcohol addiction, other conditions associated with substance abuse, and spasticity.

18. The method of claim 13 wherein the protection of the ganglion cells against glutamate-induced cytotoxicity protects the ganglion cells against damage caused by a disease or condition selected from the group consisting of nausea, AIDS wasting syndrome, multiple sclerosis, cerebral palsy, epilepsy, bronchial asthma, depression, anxiety, and sleep disorders.

19. A method for protecting against glutamate-induced neurotoxicity in cells of the central nervous system (CNS) comprising increasing the activity of a cannabinoid agonist that binds specifically to either the CB₁ or the CB₂ receptor to protect the cells of the central nervous system against glutamate-induced cytotoxicity.

20. The method of claim 19 wherein the protection of CNS cells against glutamate-induced cytotoxicity protects against damage caused by a disease or condition selected from the group consisting of stroke, hypoxia, focal or global ischemia, global hypoxic ischemic insults, Huntington's chorea, Parkinson's disease, Alzheimer's disease, hyperglycemia, diabetes, traumatic injury, CNS trauma, cardiac arrest, macular degeneration, mental diseases, schizophrenia, AIDS dementia complex, other dementias, opioid addiction, cocaine addiction, alcohol addiction, other conditions associated with substance abuse, and anorexia.

21. The method of claim I wherein the step of increasing the activity of the cannabinoid agonist comprises blocking degradation of naturally-occurring endogenous cannabinoid agonists in the cells.

22. The method of claim 21 wherein the blockage of the naturally-occurring endogenous cannabinoid agonists occurs by inhibition of anandamide amidohydrolase.
